(19) United States
(12) Patent Application Publication
(10) Pub. No.: US 2023/0104961 A1
(43) Pub. Date: Apr. 6, 2023

(54) DETECTION DEVICE AND DETECTION METHOD OF SUBSTANCE TO BE DETECTED USING SURFACE-ENHANCED RAMAN SCATTERING

(71) Applicant: KOREA RESEARCH INSTITUTE OF STANDARDS AND SCIENCE, Daejeon (KR)

(72) Inventors: Eun-Ah YOU, Daejeon (KR); Wansun KIM, Daejeon (KR); Tae Geol LEE, Daejeon (KR)

(21) Appl. No.: 16/982,960

(22) PCT Filed: Sep. 18, 2019
(86) PCT No.: PCT/KR2019/012074

§ 371 Date: Sep. 21, 2020

(30) Foreign Application Priority Data
Sep. 5, 2019 (KR) 10-2019-0110220

(51) Int. Cl. G01N 33/68; G01N 21/65; G01N 21/64; G01N 33/487; G01N 33/543

(52) U.S. Cl. CPC G01N 33/6848; G01N 21/658; G01N 21/6428; G01N 33/487; G01N 33/54373; G01N 2021/6439

(57) ABSTRACT

Provided are a detection method and a detection device of a substance using surface-enhanced Raman scattering according to the present invention, the detection method according to the present invention including: bringing Raman-active particles into contact with a sample which may contain a substance to be detected, the Raman-active particle including a spherical plasmonic metal core, a plasmonic metal shell having surface unevenness, a self-assembled monolayer which is bonded to each of the core and the shell and positioned between the core and the shell, and includes a Raman reporter, and a first receptor which is positioned on a surface of the plasmonic metal shell and may be specifically bonded to the substance to be detected; and irradiating excitation light thereon to detect the substance to be detected in the sample.

### TECHNICAL FIELD

The present invention relates to a detection device and a detection method of a substance to be detected using surface-enhanced Raman scattering, and particularly, to provide a detection device and a detection method having excellent reliability and reproducibility and allowing detection at a single molecule level.

### BACKGROUND ART

Surface-enhanced Raman scattering (hereinafter, referred to as SERS) spectrometry is spectrometry using a phenomenon in which a Raman scattering intensity rapidly increases 10⁶ to 10⁸ times or more when molecules are adsorbed on a surface of a metal nanostructure such as gold and silver. The SERS spectrometry fused with nanotechnology, which currently develops at a very rapid pace, is particularly expected a lot to be critically used as a medical sensor.

As an example, since the SERS spectrometry is measurement technology having a high selectivity and high informativity, and simultaneously, is a powerful analysis method for chemical/biological/biochemical analysis of ultrahigh sensitivity, a study for performing early diagnosis of various diseases including Alzheimer's disease, diabetes, or the like, together with high-sensitivity DNA analysis, using SERS spectroscopy, is currently being actively conducted.

However, though SERS spectrometry has high selectivity, high informativity, and high sensitivity, signal enhancement changes very sensitively depending on the size or type of a gap or a junction between plasmon metals, a distance between a hot spot and a Raman signal generation source, and the like, and thus, reliability and reproducibility of measurement are deteriorated.

Thus, in order to be utilized in the biofields such as early diagnosis of diseases, development of a Raman-active particle which has high sensitivity to allow detection at a single molecule level and in which reliable and reproducible surface-enhanced Raman scattering occurs should be preceded, and development of technology of mass-producing the Raman-active particles within a short time should be also preceded.

### DISCLOSURE

### Technical Problem

An object of the present invention is to provide a detection device and a detection method based on surface-enhanced Raman scattering which have reproducibility of an object to be detected and reliability and allow quantitative detection.

Another object of the present invention is to provide a detection device and a detection method based on surface-enhanced Raman scattering which have extremely good sensitivity to allow detection at a single molecule level.

Still another object of the present invention is to provide a detection device and a detection method based on surface-enhanced Raman scattering which is used in a detection device and a detection method and is appropriate for detection of biomaterials including a biochemical material since elements in contact with an object to be detected has biocompatibility.

### Technical Solution

In one general aspect, a detection method according to the present invention includes: bringing Raman-active particles into contact with a sample which may contain a substance to be detected, the Raman-active particle including a spherical plasmonic metal core, a plasmonic metal shell having surface unevenness, a self-assembled monolayer which is bonded to each of the core and the shell and positioned between the core and the shell, and includes a Raman reporter, and a first receptor which is positioned on a surface of the plasmonic metal shell and may be specifically bonded to the substance to be detected; irradiating excitation light thereon to detect the substance to be detected in the sample.

In the detection method according to an exemplary embodiment of the present invention, the excitation light may be a near-infrared ray.

The detection method according to an exemplary embodiment of the present invention may include a) bringing a substrate in which a second receptor which is specifically bonded to the substance to be detected is positioned on a surface into contact with a sample; b) bringing the substrate in contact with the sample into contact with the Raman-active particles; c) irradiating the substrate in contact with the Raman-active particles with the excitation light to obtain a Raman spectrum; and d) detecting the presence of the substance to be detected in the sample and a concentration of the substance to be detected, based on the Raman spectrum.

In the detection method according to an exemplary embodiment of the present invention, step c) may include performing Raman mapping in a predetermined area to obtain a Raman map of one Raman signal; and step d) may include detecting the presence of the substance to be detected in the sample and the concentration of the substance to be detected, based on a total intensity obtained by summing up maximum intensities to the one Raman signal on the Raman map.

In the detection method according to an exemplary embodiment of the present invention, the concentration of the substance to be detected may be calculated by the following Equation 1:

MC=aIₛᵤₘ+b (Equation 1)

wherein MC is a log value of a molar concentration of the substance to be detected, Iₛᵤₘ is a total intensity, and a and b are a constant, respectively.

The detection method according to an exemplary embodiment of the present invention may further include removing an unreacted sample after step a) and removing unreacted Raman-active particles after step b).

In the detection method according to an exemplary embodiment of the present invention, the substance to be detected may be one or two or more selected from lesion biomarkers having lesion specificity, pathogens, proteins, nucleic acids, sugars, drugs, and biochemical materials.

In the detection method according to an exemplary embodiment of the present invention, basal fluorescence may not be removed from the Raman map.

In the detection method according to an exemplary embodiment of the present invention, the plasmonic metal shell may include plasmonic metal fine particles having an average size of 0.1D to 0.6D, based on a diameter (D) of the metal core, and have surface unevenness by the plasmonic metal fine particles.

In the detection method according to an exemplary embodiment of the present invention, in the plasmon metal shell, an inner shape of the shell in contact with the self-assembled monolayer may be spherical.

In the detection method according to an exemplary embodiment of the present invention, the self-assembled monolayer may have a thickness of 0.5 to 2.0 nm.

In another general aspect, a detection method of a lesion biomarker for disease diagnosis is provided.

The detection method according to the present invention is a detection method of a lesion marker for disease diagnosis, and includes: bringing a substrate in which a second receptor which is specifically bonded to the lesion marker is positioned on a surface into contact with a living body-derived sample which may contain the lesion marker; bringing the substrate in contact with the living body-derived sample into contact with Raman-active particles and then washing the substrate, the Raman-active particle including a spherical plasmonic metal core, a plasmonic metal shell having surface unevenness, a self-assembled monolayer which is bonded to each of the core and the shell and positioned between the core and the shell, and includes a Raman reporter, and a first receptor which is positioned on a surface of the plasmonic metal shell and may be specifically bonded to the substance to be detected; irradiating the substrate in contact with the Raman-active particles with near-infrared excitation light and performing Raman mapping in a predetermined area to obtain a Raman map for the one Raman signal; and detecting the presence of the lesion marker in the living body-derived sample and a concentration of the lesion marker, based on a total intensity obtained by summing up maximum intensities to the one Raman signal on the Raman map.

In another general aspect, a detection device for detecting a substance to be detected using surface-enhanced Raman scattering is provided.

The detection device according to the present invention includes: a surface-enhanced Raman scattering-active reagent including Raman-active particles which include a spherical plasmonic metal core, a plasmonic metal shell having surface unevenness, a self-assembled monolayer which is bonded to each of the core and the shell and positioned between the core and the shell, and includes a Raman reporter, and a first receptor which is positioned on a surface of the plasmonic metal shell and may be specifically bonded to the substance to be detected; and a substrate in which a second functional group which is specifically bonded to a substance to be detected is positioned on a surface.

### Advantageous Effects

The Raman-active particle used in the detection method and the detection device according to an exemplary embodiment of the present invention has a core-shell structure of a spherical plasmon active core and a plasmon active shell having surface unevenness due to fine particles. In addition, the Raman-active particle has hot spots which have strictly defined size and shape and are uniformly present in the whole area of the particle, by the self-assembled monolayer including a Raman reporter positioned between the core and the shell, the spherical core shape, and the shell surrounding the self-assembled monolayer to have a spherical inner surface. In addition, the Raman-active particle has a Raman reporter positioned uniformly at a high density in the form of the self-assembled monolayer, in the area of the hot spots which are well defined and present uniformly and continuously in the whole area of the particle. In addition, since in the metal shell, metal fine particles themselves protrude and form bumpy unevenness on the whole area of a metal shell surface, the Raman activity of one particle is uniform and the Raman activity between particles is also uniform, while the sensitivity of particles may be greatly improved. Due to the characteristics and the structure of the Raman-active particles, the Raman-active particles have uniform Raman activity based on the particles, and also, the Raman activity between particles is substantially almost the same. Thus, the detection method and the detection device using the Raman-active particles allow reproducible and reliable quantitative detection of the substance to be detected, and may have excellent detectability at a single molecule level.

In addition, well-defined hot spots are continuously present in a whole area of the Raman-active particle, and the Raman reporter is uniformly positioned at a high density in the well-defined hot spot. Thus, the detection method and the detection device using the Raman-active particles allow a biochemical material (biomaterial) having a several or several tens of micrometers in size to be reproducibly detected also.

In addition, the detection method and the detection device according to an exemplary embodiment of the present invention allow detection of a material by near-infrared irradiation of 750 nm or more and may prevent damage of a biological sample by excitation light irradiation for Raman analysis.

In addition, the detection method and the detection device according to an exemplary embodiment of the present invention have a high Raman signal intensity and simultaneously hardly produce basal fluorescence, when a near-infrared ray of 750 nm or more is irradiated as excitation light, and thus, the particle is free from Raman signal distortion due to post-processing of a detection signal to have high detection reliability.

In addition, the Raman-active particle used in the detection method and the detection device according to an exemplary embodiment of the present invention is free from a surfactant, and thus, has a characteristic of having biocompatibility.

In addition, in the Raman-active particle used in the detection method and the detection device according to an exemplary embodiment of the present invention, an organic constituent component including a Raman reporter is protected by being wrapped by a shell, and the self-assembled monolayer-metal shell of a core-Raman reporter is strongly bonded by a functional group, and thus, the Raman-active particle has very good durability and physical/chemical stability.

In addition, the detection device according to an exemplary embodiment of the present invention allows mass production of the Raman-active particles within a short time by an extremely simple method of mixing a buffer solution, a metal precursor, and a spherical core metal at room temperature, and thus, has excellent commerciality.

### DESCRIPTION OF DRAWINGS

The above and other objects, features and advantages of the present invention will become apparent from the following description of preferred embodiments given in conjunction with the accompanying drawings, in which:

FIG. 1 is scanning electron micrographs of Raman-active particles produced according to an exemplary embodiment of the present invention at a low magnification (a) and at a high magnification (b).

FIG. 2 is transmission electron micrographs of the Raman-active particles produced according to an exemplary embodiment of the present invention.

FIG. 3 is a drawing illustrating a UV-Vis absorption spectrum of each of an Au core itself (cpre AuNP in FIG. 3), an Au core on which a self-assembled monolayer is formed (BDT-treated AuNP in FIG. 3), and the Raman-active particles produced according to an exemplary embodiment of the present invention.

FIG. 4 is a drawing illustrating a measured Raman spectrum of the Raman-active particles themselves produced according to an exemplary embodiment of the present invention.

FIG. 5 is a scanning electron micrograph (a) of the Raman-active particles (A, B, and C) produced according to an exemplary embodiment of the present invention observed after positioning the particles on a silicon substrate, and a drawing illustrating Raman mapping (laser at 780 nm, 5 mW) (b) in an area observed by a scanning electron microscope.

FIG. 6 is a drawing illustrating a Raman spectrum of each of the Raman-mapped Raman-active particles (A, B, and C) in FIG. 5 by overlapping.

FIG. 7 is illustrating a Raman spectrum (a) and a Raman map (b) obtained according to an exemplary embodiment of the present invention, with tau protein as a substance to be detected.

FIG. 8 is a schematic diagram illustrating a structure in which tau protein is bonded to a second receptor of a tau substrate and a tau probe is bonded to the tau protein bonded to a substrate, according to an exemplary embodiment of the present invention.

FIG. 9 is a drawing illustrating Raman mapping results depending on a tau protein concentration in a sample, according to an exemplary embodiment of the present invention.

FIG. 10 is a drawing illustrating a reference graph calculated from a standard sample with a different tau protein concentration (Tau in PBS in FIG. 10) and results of detecting a cerebrospinal fluid collected from a human body (Tau in aCSF in FIG. 10) or a plasma (Tau in plasma in FIG. 10) as samples, according to an exemplary embodiment of the present invention.

### BEST MODE

Hereinafter, the detection method and the detection device of the present invention will be described in detail with reference to accompanying drawings. The drawings to be provided below are provided by way of example so that the idea of the present invention can be sufficiently transferred to a person skilled in the art to which the present invention pertains. Therefore, the present invention is not limited to the drawings provided below but may be embodied in many different forms, and the drawings suggested below may be exaggerated in order to clear the spirit of the present invention. Technical terms and scientific terms used herein have the general meaning understood by those skilled in the art to which the present invention pertains unless otherwise defined, and a description for the known function and configuration which may unnecessarily obscure the gist of the present invention will be omitted in the following description and the accompanying drawings. In addition, the singular form used in the specification and claims appended thereto may be intended to also include a plural form, unless otherwise indicated in the context. Units used in the present specification and attached claims thereto without particular mention are based on weights, and as an example, a unit of % or ratio refers to a wt % or a weight ratio.

In the detection method according to the present invention, Raman-active particles are brought into contact with a sample which may contain a substance to be detected, and excitation light is irradiated thereon, thereby detecting the substance to be detected in the sample.

In a specific example, the Raman-active particle is a Raman-active particle for surface-enhanced Raman scattering (SERS) and includes: a spherical plasmonic metal core; a plasmonic metal shell having surface unevenness; a self-assembled monolayer which is bonded to each of the core and the shell and positioned between the core and the shell, and includes a Raman reporter; and a first receptor which is positioned on a surface of the plasmonic metal shell and may be specifically bonded to the substance to be detected.

A self-assembled monolayer positioned between a core and a shell is a strictly adjusted thickness due to the characteristics of self assembly. Thus, a strictly defined nanogap having a size corresponding to the thickness of the self-assembled monolayer may be formed between the core and the shell. In addition, nanogaps (hot spots) having a uniform size may be formed in a whole area of the Raman-active particle by the structure of core-self-assembled monolayer-shell.

In addition, since the shape of the plasmonic metal core is spherical, the self-assembled monolayer has a spherical shape, and in the plasmonic metal shell, the inner shape of the metal shell in contact with the self-assembled monolayer may also have a spherical shape. Thus, nanogaps (hot spots) may be positioned in the whole area of the Raman-active particle, and simultaneously, the nanogaps (hot spots) may be positioned in the same well-defined position in all directions based on a radiation direction.

In addition, since in the Raman-active particle, the self-assembled monolayer positioned between the core and the shell contains the Raman reporter, the Raman reporter is positioned in the well-defined and same position in the radiation direction in the Raman-active particle, the Raman reporter is positioned uniformly at a high density in the whole area of the Raman-active particle, and also, the Raman reporter is positioned in the hot spot.

The Raman-active particle may represent uniform SERS activity based on the particle, have little deviation of the Raman activity between particles to represent uniform SERS activity between particles, and achieve larger Raman signal enhancement.

As a specific example of uniform SERS activity, a standard deviation (a.u.) of a Raman signal intensity (a maximum intensity of one Raman signal, a.u.) on a Raman spectrum of the Raman-active particles may be 8.0 or less. Here, the Raman spectrum of the Raman-active particles may be obtained by irradiating near-infrared light, using a known kind of Raman spectroscope. As an example, Raman spectroscopic analysis may be performed under the conditions of a laser at 780 nm, a laser power of 6 mW, an N.A. 0.75 object lens, and a laser exposure time of 1 second. The standard deviation may be calculated from the Raman spectrum of 50 or more Raman-active particles, but is not necessarily limited thereto.

As a specific example, each of the plasmon metal core and the plasmon metal shell may be a metal generating surface plasmon by an interaction with light. As an example, each of the plasmon metal core and the plasmon metal shell may be gold, silver, platinum, palladium, nickel, aluminum, copper, a mixture thereof, an alloy thereof, or the like. However, each of the plasmon metal core and the plasmon metal shell may be gold or silver, considering biocompatibility.

As a specific example, the plasmonic metal shell may include plasmonic metal fine particles having an average size of 0.1D to 0.6D, based on a diameter (D) of the metal core, and may have surface unevenness due to the plasmonic metal fine particles. Specifically, the metal shell in the state of being bonded to the self-assembled monolayer may be composed of metal fine particles having an average size of 0.1D to 0.6D, based on the diameter (D) of the metal core, and the metal shell may have irregular unevenness due to the particle shape of the metal fine particles.

An unevenness structure due to the metal fine particles of the plasmonic metal shell may form the hot spots on the surface of the shell itself, together with the hot spots by the nanogaps of the metal core and the metal shell, and thus, is more advantageous for signal enhancement. In addition, since in the metal shell, the metal fine particles themselves protrude to form bumpy unevenness in the whole area of the metal shell, the sensitivity of the Raman-active particle may be increased by the metal shell, and also uniformity of Raman activity in one particle and uniformity of Raman activity between particles may not be inhibited.

An average diameter of the plasmon metal core may be in the level of 20 to 100 nm, specifically 20 to 80 nm, and more specifically 30 to 70 nm, but is not limited thereto.

In a specific example, the self-assembled monolayer may be a self-assembled monolayer of the Raman reporter. The Raman reporter may refer to an organic compound (organic molecule) including a Raman-active molecule, or an organic compound (organic molecule) having a bonding force to the metal of the metal core and including a Raman-active molecule. The Raman reporter is previously known in the art, and may be any one as long as it is widely used in the art.

Since the Raman reporter (molecule) has a bonding force to the metal of the metal core, the self-assembled monolayer of the Raman reporter may be formed on the metal core where a pure metal surface is exposed.

The Raman-active molecule may include a surface-reinforced Raman-active molecule, a surface-enhanced resonance Raman-active molecule, a hyper-Raman-active molecule, or a coherent anti-stokes Raman-active molecule. The Raman-active molecule may have both a Raman signal and a fluorescent signal, or have a Raman signal.

As a specific example, the Raman-active molecule may be selected from a group consisting of cyanines, fluorescein, rhodamine, 7-nitrobenz-2-oxa-1,3-diazole (NBD), phthalic acids, terephthalic acids, isophthalic acids, cresyl fast violet, cresyl blue violet, brilliant cresyl blue, paraaminobenzoic acid, erythrosin, biotin, dioxigenin, phthalocyanine, azomethine, xanthine, N,N-diethyl-4-(5′-azobenzotriazolyl)-phenylamine, aminoacridine, and a combination thereof. Examples of cyanine may include Cy3, Cy3.5, or Cy5. Examples of fluorescein may include carboxyfluorescein (FAM), 6-carboxy-2′,4,4′,5′,7,7′-hexachlorofluorescein (HEX), 6-carboxy-2′,4,7,7′-tetrachlorofluorescein (TET), 5-carboxy-4′,5′-dichloro-2′,7′-dimethoxyflulorescein, 6-carboxy-4′,5′-dichloro-2′-7′-dimethoxyfluorescein (Joe), 5-carboxy-2′,4′,5′,7′-tetrachlorofluorescein, 5-carboxyfluorescein, or succinylfluorescein. Examples of rhodamine may include tetramethylrhodamine (Tamra), 5-carboxyrhodamine, 6-carboxyrhodaminerhodamine, 6G (rhodamine 6G: R6G), tetramethyl rhodamine isothiol (TRIT), sulforhodamine 101 acid chloride (Texas Red dye), carboxy-X-rhodamine (ROX), or Rhodamine B.

As another specific example, the Raman-active molecule may be a Raman-active molecule in the form of a benzene ring, and the Raman-active molecule in the form of a benzene ring may include 4-aminothiophenol (4-ATP), 4-mercaptobenzoic acid (4-MBA), phenyl isothiocyanate (PITC), benzenethiol (BT), 1,4-benzenedithiol (BDT), biphenyl-4,4′-dithiol (BPDT), p-terphenyl-4,4″-dithiol (TPDT), 4-bromobenzenethiol (4-BBT), 4-chlorobenzenethiol (4-CBT), 3,3′-diethylthiatricarbocyanine iodide (DTTC), and the like.

However, since nanogaps (hot spots) are formed between the metal core and the metal shell by the Raman reporter bonded to the metal core, a length (size) of the Raman reporter may be 3 nm or less, specifically 0.5 to 2 nm, in terms of forming hot spots where stronger signal enhancement is done. Here, the length (size) of the Raman reporter corresponds to the thickness of the self-assembled monolayer, of course.

In addition, the Raman reporter includes the Raman-active molecule, but preferably has a first functional group which is spontaneously bonded to the metal core. More preferably, the Raman reporter has the first functional group which is spontaneously bonded to the metal of the metal core and a second functional group which is spontaneously bonded to the metal of the metal shell. In this case, the self-assembled monolayer is bonded to each of the metal core and the metal shell, thereby greatly improving a bonding force between the metal shell and the metal core to which the Raman reporter is fixed.

The functional group (the first functional group or the second functional group) may be any functional group as long as it is spontaneously bonded to the corresponding metal, considering the metals of the core and the shell. As a substantial example, when a first metal and a second metal are independently of each other gold or silver, the functional group (the first functional group or the second functional group) may be a thiol group (—SH), a carboxyl group (—COOH), an amine group (—NH₂), or the like, but the present invention is not limited to the specific kinds of functional group.

The Raman-active molecule having a bonding force to the metal of the metal core by the first functional group is spontaneously bonded (fixed) to the metal core, whereby the self-assembled monolayer of the Raman reporter may be formed on the metal core, and a film of the Raman reporter having a uniform thickness may be homogeneously formed on the whole surface of the metal core.

The Raman-active particle may include a receptor which is fixed to the plasmonic metal shell and may be specifically bonded to the substance to be detected. The receptor may be any material known to be specifically bonded to the substance to be detected, such as complementary bonding between enzyme-substrate, antigen-antibody, protein-protein or DNA-DNA. Here, the receptor may include a functional group which is spontaneously bonded to the metal of the metal shell (as an example, a thiol group, a carboxyl group, an amine group, or the like), and may be in the state of being spontaneously and chemically bonded to the metal shell by the functional group. If necessary, the metal shell may react with the functional group of the receptor and be bonded thereto, or may be in the state of being surface-modified so as to be bonded to a blocking molecule described later. As an example, the surface of the metal shell may be a surface modified with a carboxyl group, a thiol group, an amine group, a hydroxyl group, and the like.

In a specific example, the Raman-active particle may further include a blocking molecule covering a surface area of the shell to which the receptor is not attached (bonded). The blocking molecule prevents an undesired interaction between the substance to be detected and the shell surface itself, not the receptor, and may serve to make orientation of the receptor positioned on the surface of the shell more constant. The blocking molecule may be any material which is commonly used for preventing nonspecific bonding on the bare metal surface in the biosensor field, such as bovine serum albumin (BSA).

The substance to be detected may be a material derived from a living (including a virus) or non-living thing. Specifically, the substance to be detected may include a lesion biomarker having lesion specificity, a pathogen, a protein, a nucleic acid, a sugar, a drug, a biochemical material, and the like. More specifically, the substance to be detected may be an amino acid, a peptide, a polypeptide, a protein, a glycoprotein, a lipoprotein, a nucleoside, a nucleotide, an oligonucleotide, a nucleic acid, a sugar, a carbohydrate, an oligosaccharide, a polysaccharide, a fatty acid, a lipid, a hormone, a metabolite, a cytokine, a chemokine, a receptor, a nucleotransmitter, an antigen, an allergen, an antibody, a substrate, a metabolite, a cofactor, an inhibitor, a drug, a pharmaceutical material, a nutritional substance, a prion, a toxin, a poisonous material, an explosive material, an insecticide, a chemical weapon agent, a biohazard agent, a radioactive isotope, a vitamin, a heterocyclic aromatic compound, a carcinogen, a mutagen, an anesthetic, an amphetamine, a barbiturate, a hallucinogen, waste, or a pollutant. In addition, when the analyte is a nucleic acid, the nucleic acid may include genes, viral RNA and DNA, bacterial DNA, fungal DNA, mammalian DNA, cDNA, mRNA, RNA and DNA fragments, oligonucleotides, synthetic oligonucleotides, modified oligonucleotides, single- and double-stranded nucleic acids, natural and synthetic nucleic acid, and the like.

The substance to be detected may be positioned in-vivo, and may be detected in-vivo. That is, the Raman-active particle described above may be for use in-vivo, and for biological injection.

On the contrary, the substance to be detected may be positioned in-vitro, and may be detected in-vitro. That is, the Raman-active particle described above may be used in-vitro. Here, the substance to be detected may be in the form of a sample collected in-vivo such as blood, urine, mucosal detachment, saliva, body fluids, tissues, biopsy materials, a combination thereof, or the like, but is not limited thereto.

In a specific example, the Raman-active particle may be for near-infrared excitation light having a wavelength of 750 nm or more, specifically near-infrared excitation light in a wavelength region of 750 to 1500 nm, and more specifically, in a wavelength region of 750 to 1000 nm, a wavelength region of 770 nm to 1500 nm, or a wavelength region of 780 nm to 1000 nm. That is, the Raman-active particle allows detection and analysis of the substance to be detected by light irradiation in a near-infrared region.

As is known, when visible light is irradiated on a biomaterial including a biochemical material, a fluorescence phenomenon may occur. Since fluorescence intensity is very strong as compared with Raman scattering and fluorescence occurs in a similar region to Raman scattering, it is difficult to obtain pure Raman spectrum covered with a fluorescence peak. Therefore, SERS analysis by light irradiation in a near-infrared region, not a visible region, may obtain a Raman spectrum without an influence of fluorescence, and thus, is very advantageous in a biofield.

Substantially, when the substance to be detected is detected using the Raman-active particles according to a specific example, basal fluorescence may not be substantially shown on a Raman spectrum of the substance to be detected obtained by near-infrared irradiation.

Thus, fluorescence may not be removed from the Raman spectrum obtained by near-infrared irradiation. That is, the Raman spectrum used for detection of the presence of the substance to be detected and the quantitative analysis of the substance to be detected may be a spectrum detected in a Raman detector or a spectrum which is detected in a Raman detector and is not modified by post-treatment.

However, the Raman-active particle of the present invention should not be interpreted limitedly as being used for a near-infrared ray, and excitation light irradiated in a detection method of the present invention should not be interpreted limitedly as being a near-infrared ray. As an example, based on the center wavelength (λₘₐₓ) of a maximum absorption peak in a UV-visible light absorption spectrum of the Raman-active particles, light in a wavelength region of the center wavelength (λₘₐₓ)±150 nm may be irradiated as the excitation light, and in this case, a Raman spectrum in which larger Raman signal enhancement is formed may be obtained. In an exemplary embodiment, light in a wavelength region of 500 to 750 nm, 500 to 750 nm, 550 to 700 nm, or 600 to 680 nm may be irradiated as excitation light.

As described above, in the detection method according to the present invention, the Raman-active particles are brought into contact with a sample which may contain the substance to be detected, and excitation light is irradiated thereon, thereby detecting the substance to be detected. Specifically, the detection method according to the present invention may include bringing the Raman-active particles into contact with the sample which may contain the substance to be detected and irradiating excitation light thereon to obtain a Raman spectrum; and detecting (quantitatively analyzing) the substance to be detected in the sample based on the obtained Raman spectrum.

As a specific example, the excitation light may be light in a region of visible to near-infrared ray. As described above, when the excitation light is a near-infrared ray, the excitation light may be a near-infrared ray in a wavelength region of 750 nm to 1500 nm, a wavelength region of 750 nm to 1000 nm, a wavelength region of 770 nm to 1500 nm, or a wavelength region of 780 nm to 1000 nm. On the contrary, when the excitation light is light in a visible region, the excitation light may be visible light in a wavelength region of the center wavelength (λₘₐₓ)±150 nm, a wavelength region of the center wavelength (λₘₐₓ)±100 nm, or a wavelength region of the center wavelength (λₘₐₓ)±50 nm, based on the center wavelength (λₘₐₓ) of a maximum absorption peak in a UV-visible light absorption spectrum of the Raman-active particles.

As a specific example, the detection method may include a first step of bringing a sample into contact with an active surface of a substrate which provides the active surface on which a second receptor which may be specifically bonded to the substance to be detected is formed; a second step of bringing the Raman-active particles into contact with the active surface in contact with the sample; a third step of irradiating the active surface in contact with the Raman-active particles with excitation light to obtain a Raman spectrum; and a fourth step of detecting the presence of the substance to be detected in the sample and a concentration of the substance to be detected, based on the Raman spectrum. The second receptor may be specifically bonded to the substance to be detected, and when the substance to be detected is present in the sample, the substance to be detected may be fixed to the active surface by the second receptor. Here, the first receptor and the second receptor may be specifically bonded to different sites from each other of the substance to be detected, of course.

The active surface of the substrate may be the surface of a metal such as gold, silver, platinum, palladium, nickel, aluminum, or copper, but is not limited thereto. However, when the sample is a living body-derived sample or a biosample, the metal may be gold, silver, platinum, or the like having biocompatibility. The receptor may include a functional group which is spontaneously bonded to the active surface (as an example, a thiol group, a carboxyl group, an amine group, or the like), and may be in the state of being chemically bonded to the active surface by the functional group. If necessary, the active surface may react with the functional group of the second receptor and be bonded thereto, or may be in the state of being surface-modified so as to be bonded to a blocking molecule described later. As an example, the active surface may be a surface modified with a carboxyl group, a thiol group, an amine group, a hydroxyl group, and the like.

Contacting in the first step may be performed by applying a liquid sample (liquid sample or liquefied sample) on the active surface or immersing the active surface in the liquid sample. After a sufficient time for the substance to be detected which may be present in the sample to be stably bonded to the second receptor has passed, the applied liquid sample may be removed.

Contacting in the second step may be performed by applying the Raman-active particle dispersion solution on the active surface in contact with the sample, or immersing the active surface in the Raman-active particle dispersion solution. After a sufficient time for the Raman-active particles to be stably bonded to the substance to be detected fixed to the active surface has passed, unreacted Raman-active particles may be removed.

When the sample is applied and the Raman-active particle dispersion solution is applied, a predetermined amount may be applied at each time, and the applied Raman-active particle dispersion solution may contain the Raman-active particles at a constant concentration, of course. In addition, when a disperse medium of the Raman-active particle dispersion solution or the sample is liquefied, a solvent or disperse medium used for liquefaction does not chemically react with the Raman-active particles or the sample, and may be any material as long as it does not affect Raman measurement. When a living body-derived sample or a biomaterial is analyzed, an example of a material used for liquefaction of the disperse medium or the sample of the Raman-active particles may include a buffer solution such as a HEPES solution, deionized water, and the like, but the present invention is not limited thereto.

Removal of the unreacted liquid sample (residual liquid sample) or removal of the unreacted Raman-active particles (residual Raman-active particles) applied on the active surface may be performed by washing the active surface using a washing solution which does not adversely affect a substance to be analyzed and the Raman-active particles. When the living-derived sample or the biomaterial is analyzed, an example of the washing solution used in washing may include deionized water or the like, but the present invention is not limited thereto.

By the second step, the Raman-active particles are specifically bonded to the substance to be detected fixed to the active surface, thereby forming a bonding structure of active surface-second receptor-substance to be detected-Raman-active particles.

In the third step, excitation light (irradiation light) may be light in a visible to near-infrared region, advantageously, a near-infrared ray having a wavelength of 750 nm or more, specifically a near-infrared ray having a wavelength region of 750 nm to 1500 nm, and more specifically a near-infrared ray having a wavelength of 750 nm to 1000 nm, a wavelength of 770 nm to 1500 nm, or a wavelength of 780 nm to 1000 nm.

In the fourth step, the presence of the substance to be detected in the sample and the concentration of the substance to be detected may be calculated, based on the obtained Raman spectrum. Here, the Raman spectrum obtained by near-infrared irradiation in the third step may be a spectrum from which basal fluorescence is not removed. That is, the Raman spectrum used for detection of the presence of the substance to be detected and the quantitative analysis of the substance to be detected may be a spectrum which is detected in the Raman detector and is not modified by post-treatment.

In a specific example, the third step may be a step of performing Raman mapping in a predetermined area to obtain a Raman map of one Raman signal; and the fourth step may be a step of detecting the presence of the substance to be detected in the sample and the concentration of the substance to be detected, based on a total intensity obtained by summing up maximum intensities to the one Raman signal on the Raman map.

One Raman signal (one Raman wavelength) forming the Raman map may be a signal having a highest peak intensity among the Raman spectra of the substance to be detected, or a signal having a narrowest half-width of the peak, but is not limited thereto.

The Raman mapping may be Raman mapping to an area having a predetermined size (area of the active surface), and the predetermined size may be 1 to 100 μm×1 to 100 μm, but is not limited thereto. In addition, a mapping interval in the Raman mapping may be in a level of 0.1 μm to 10 μm to each of axes perpendicular to each other, an output of excitation light (excitation laser light) may be in a level of 1 mW to 90 mW, as a practical example, 1 mW to 10 mW, an excitation light irradiation time may be 0.5 to 10 seconds, and the number of scanning may be 1 to 5, but are not limited thereto.

In the fourth step, intensities of one Raman signal present on the Raman map are summed up and the concentration of the substance to be detected may be quantified. Here, the intensities to be summed up may be a maximum intensity value of the corresponding Raman signal (a maximum intensity value of a Raman peak).

That is, quantitative analysis of the substance to be detected is possible only by summing up the intensities of certain Raman peaks (first peaks) present on the Raman map, by the Raman-active particles having isotropic SERS activity, highly uniform SERS activity between the Raman-active particles, substantially no occurrence of basal fluorescence, reliability representing an excellent Raman signal intensity, and the like, and a limit of detection is in a level of 20 aM, and thus, the sensitivity is extremely high so that even a single substance to be detected may be detected.

Substantially, in a semi-log graph in which a log value of a molar concentration of the substance to be detected is represented in the x-axis, and the sum of Raman signal intensities (a.u.) of certain Raman peaks (first peaks) is represented in the y-axis, the long value of the molar concentration (MC) of the substance to be detected and the sum of the Raman signal intensities (Iₛᵤₘ) are in a straight line relationship. That is, in the semi-log graph, MC=aIₛᵤₘ+b (each of a and b is a constant. Here, this linearity may be maintained in a wide range of molar concentrations of 10⁻² fM to 10⁶ fM.

Thus, the detection method may further include, before the first step, performing the Raman mapping using standard samples containing the substance to be detected at a predetermined molar concentration and obtaining the sum of Raman signal intensities in a certain Raman signal at the corresponding concentration on the Raman map, thereby obtaining a reference graph which is a relation between the molar concentration of the substance to be detected and the sum of the Raman signal intensities, on a semi-log graph. By putting the intensity calculated in the fourth step (summed intensity) on the reference graph, an amount of the substance to be detected in the sample may be quantitatively analyzed.

The present invention includes a detection method of a lesion marker for disease diagnosis. The detection method of a lesion marker may correspond to a diagnosis method of a disease using the lesion marker. In a specific example, a disease (including a disorder) may be an Infectious disease, a proliferative disease, a neurodegenerative disease, cancer, a psychological disease, a metabolic disease, an autoimmune disease, a sexually transmitted disease, a gastrointestinal disease, a lung disease, a cardiovascular disease, a stress-related disorder, a fatigue-related disorder, a fungal disease, a pathogenic disease, or an obesity-related disorder, but is not limited thereto.

The detection method of a lesion marker according to the present invention includes: bringing a substrate in which a second receptor which is specifically bonded to the lesion marker is positioned on a surface into contact with a living body-derived sample which may contain the lesion marker; bringing the substrate in contact with the living body-derived sample into contact with Raman-active particles and then washing the substrate, the Raman-active particle including a spherical plasmonic metal core, a plasmonic metal shell having surface unevenness, a self-assembled monolayer which is bonded to each of the core and the shell and positioned between the core and the shell, and includes a Raman reporter, and a first receptor which is positioned on a surface of the plasmonic metal shell and may be specifically bonded to the substance to be detected; irradiating the substrate in contact with the Raman-active particles with near-infrared excitation light and performing Raman mapping in a predetermined area to obtain a Raman map for the one Raman signal; and detecting the presence of the lesion marker in the living body-derived sample and a concentration of the lesion marker, based on a total intensity obtained by summing up maximum intensities to the one Raman signal on the Raman map.

The living body-derived sample may include, unlimitedly, blood, blood products, serum, plasma, other blood fractions, tissues, tissue extracts, urine, cerebrospinal fluid, saliva, feces, skin, hair, cheek tissues, organ tissues, breath, pleural fluid, sweat, sputum, or the like, but is not limited thereto. The living body-derived sample may be a liquid as it is, or when the living body-derived sample is not a liquid, it may be liquefied. The liquefied living body-derived sample may further include an enzyme or protease which is commonly used in the treatment of the corresponding sample, protein, a compound or preservative for treating a sample collected in vivo and increasing a half-life, a chemical stabilizer, a diluent, a buffer, an additive, a cleaning agent, a lipid, a sugar, a carbohydrate, or any combination thereof. As an example, an enzyme such as mucin or protease may dissolve the collected sample.

The lesion marker may be any atom, chemical material, molecule, compound, or aggregate which is previously used for determining whether a subject from whom the living body-derived sample was collected has a disease or not, or previously used for determining a disease progression degree. As an example, the lesion marker may be an amino acid, a peptide, a polypeptide, a protein, a glycoprotein, a lipoprotein, a nucleoside, a nucleotide, an oligonucleotide, a nucleic acid, a sugar, a carbohydrate, a fatty acid, a lipid, a hormone, a metabolite, a neurotransmitter, an antibody, or the like.

The Raman spectrum may be obtained using a common Raman detection device. As a non-limiting example, the excitation light passes through a confocal optical instrument and a microscope lens to be focused on the active surface. When the substance to be detected is present on the active surface, Raman light emitted from the substance to be detected may be focused by the microscope lens and the confocal optical instrument and combined with a monochromatic light device for spectrum separation. The Raman signal may be detected by a Raman detector connected by an interface to a computer in which signals are counted and digitalized.

The present invention includes a detection device including the Raman-active particles described above.

The detection device according to the present invention is a detection device of a substance to be detected using surface-enhanced Raman scattering, and includes: a surface-enhanced Raman scattering-active reagent including Raman-active particles which include a spherical plasmonic metal core, a plasmonic metal shell having surface unevenness, a self-assembled monolayer which is bonded to each of the core and the shell and positioned between the core and the shell, and includes a Raman reporter, and a first receptor which is positioned on a surface of the plasmonic metal shell and may be specifically bonded to the substance to be detected; and a substrate in which a second functional group which is specifically bonded to the substance to be detected is positioned on a surface (active surface).

In a specific example, a blocking molecule covering a surface area to which a receptor (first receptor or second receptor) is not attached (bonded) may be further included on the substrate surface (active surface) or the metal shell surface. The blocking molecule prevents an undesired interaction between the surface of the substrate or the metal shell, not the receptor, and the substance to be detected, and may serve to make orientation of the receptor positioned on the surface of the substrate or the metal shell more constant. The blocking molecule may be any material which is commonly used for preventing nonspecific bonding on the metal surface in the biosensor field, such as bovine serum albumin (SBA).

The present invention includes a method of producing the Raman-active particles. Hereinafter, the production method according to the present invention will be described in detail. Here, the metal core, the Raman reporter, the self-assembled monolayer, the metal shell, the substance to be detected, the receptor, and the like are similar or identical to those described above for the Raman-active particle. Thus, the method of producing Raman-active particles according to the present invention includes all described above for the Raman-active particle.

The method of producing Raman-active particles according to the present invention is a method of producing Raman-active particles for surface-enhanced Raman scattering (SERS). The production method according to the present invention includes: a) forming a self-assembled monolayer including a Raman reporter in a spherical plasmonic metal core; and b) using a reaction solution in which a buffer solution, a metal core on which the self-assembled monolayer is formed, and a plasmonic metal precursor are mixed, to form a plasmonic metal shell which surrounds the metal core on which the self-assembled monolayer is formed and has surface unevenness.

The method of producing Raman-active particles according to the present invention may mass-produce Raman-active particles having reproducibility and reliability, a sensitivity allowing a single molecule detection, and biocompatibility without a separate post-treatment at low cost, by a simple process.

As is known, for metal nanogranulation and designed shaping, an organic surfactant which may suppress growth, derive growth in a certain direction, and/or stabilize nanoparticles while providing appropriate reducibility is used in a well-known or commonly used art, and also, an organic acid or an organic acid which may substitute a surfactant is used. However, the metal nanoparticles synthesized by the method have an organic surfactant which is harmful to a living body and may affect a biochemical material, bonded thereto. Thus, in order to be used in the biofield, a post-treatment process such as capping particles by a capping material having biocompatibility or substituting a harmful surface functional group of an organic surfactant or the like with another functional group having biocompatibility is necessarily required.

However, the capping using a capping material may greatly decrease the intensity of biosensing or bioimaging based on SERS spectroscopy, and when the organic surfactant is to be substituted with a biocompatible functional group, it is difficult to completely substitute the organic surfactant which is bonded to a metal material with a very strong bonding force, and thus, toxicity still remains.

Since in the method of producing Raman-active particles according to the present invention described above, a self-assembled monolayer having a Raman reporter is formed on a metal core having a bare metal surface, and then a buffer solution which already has biocompatibility and a solution containing a metal precursor are used to form a metal shell, the Raman-active particle produced is free from the organic surfactant which is harmful to a living body to have biocompatibility immediately after production.

Accordingly, in the method of producing Raman-active particles according to an exemplary embodiment of the present invention, the reaction solution may not contain a surfactant (organic surfactant), and furthermore, the reaction solution may not contain both the surfactant and the organic acid.

In addition, since in the method of producing Raman-active particles according to the present invention, the Raman-active particles are produced using a simple process of attaching the Raman reporter to the metal core and forming the metal shell using the buffer solution and the solution containing a metal precursor, the Raman-active particles may be mass-produced within a short time at low cost, and thus, the method has excellent commerciality.

In addition, since in the method of producing Raman-active particles according to the present invention, an organic material including the Raman reporter is not exposed to the surface of the Raman-active particle, but is surrounded by the metal shell, the organic substance to be detected including the Raman-active particle may be stably protected from an external environment.

In a specific example, a step of forming the self-assembled monolayer including the Raman reporter on the metal core (step (b)) may include preparing a mixed solution containing the metal core and the Raman reporter and ultrasonically stirring the solution.

Specifically, step a) may include a1) mixing the metal core and the Raman reporter so that the molar concentrations thereof are 0.01 to 1 nM and 10 to 1000 μM, to prepare a mixed solution; a2) ultrasonically stirring the solution to perform a reaction at room temperature for 10 to 30 minutes; and a3) separating and recovering the metal core to which the Raman reporter is fixed. Here, the mixed solution may be an aqueous mixed solution, but is not necessarily limited thereto.

After performing step a), b) forming the metal shell surrounding the metal core to which the Raman reporter is fixed from a reaction solution in which the buffer solution, the metal core to which the Raman reporter is fixed (metal core on which the self-assembled monolayer is formed), and the metal precursor, may be performed. The metal core to which the Raman reporter is fixed may be a metal core on which the self-assembled monolayer of the Raman reporter is formed.

In step b), the mole ratio of a buffer of the buffer solution and the metal precursor (mole ratio obtained by dividing the number of moles of the buffer by the number of moles of the metal precursor) may be 10 to 100, preferably 20 to 80. When the mole ratio is controlled to 10 to 100, preferably 20 to 80, a thin metal shell which completely surrounds the Raman reporter fixed to the metal core may be formed, and a metal shell having surface unevenness by metal fine particles may be formed.

The buffer solution may include one or more selected from 4-(2-hydroxyethyl)-1-piperazineethanesulfonic acid (HEPES), 4-(2-hydroxyethyl)piperazine-1-ethanesulfonic acid (MES), phosphated buffered saline (PBS), tris(2-amino-2-hydroxymethylpropane-1,3-idol), phosphate buffer (PB), 3-(N-morpholino)propanesulfonic acid (MOPS), 3-[[1,3-dihydroxy-2-(hydroxymethyl)propan-2-yl]amino]propane-1-sulfonic acid (TAPS), and piperazine-N,N′-bis(2-ethanesulfonic acid) (PIPES). The buffer of the buffer solution may serve as a weak reducing agent which reduces a metal, and allows a surfactant for stabilizing the produced Raman-active particles by the buffer of the buffer solution to be excluded.

The metal of the metal precursor may be gold, silver, platinum, palladium, nickel, aluminum, copper, a mixture thereof, an alloy thereof, or the like. However, the metal of the metal precursor may be preferably gold or silver, independently of the metal of the metal core, considering biostability. The metal precursor according to an advantageous example may be a gold precursor such as HAuCl₄, HAuBr₄, NaAuCl₄, AuCl₃.3H₂O, NaAuCl₄.2H₂O, or a mixture thereof, or a silver precursor such as AgNO₃, but is not limited thereto.

In a specific example, in step b), the buffer solution, the metal precursor solution, and the metal core dispersion to which the Raman reporter is fixed are mixed to prepare the reaction solution, and the reaction is performed at a temperature of 15 to 40′C, specifically a temperature of 15 to 35° C., more specifically a temperature of 15 to 25° C., and still more specifically at room temperature (21 to 25° C.). The metal shell may be prepared by reacting the reaction solution for 10 minutes to 50 minutes, specifically 20 minutes to 40 minutes, but the present invention is not limited to the reaction time of the reaction solution. Here, stirring may be performed during the reaction, and the reaction may be completed by adding an excessive amount of water to the reaction solution.

The molar concentration of the buffer in the buffer solution may be 10 to 100 mM, the molar concentration of the metal precursor in the metal precursor solution may be 1 to 10 mM, and the molar concentration of the metal core in the metal core dispersion to which the Raman reporter is fixed may be 0.01 to 0.5 nM, but are not limited thereto.

The buffer solution and the metal precursor solution may be mixed so that the mole ratio between the buffer and the metal precursor described above are satisfied, and the metal core dispersion may be mixed so that the mole ratio of the metal precursor to metal core is 1:1×10⁻⁷ to 1×10⁻⁵. Here, the metal precursor solution and the metal core dispersion are first mixed, and then the buffer solution is mixed, so that the metal shell may be uniformly formed on the metal core(s).

Specifically, step b) may include b1) mixing the metal precursor solution and the metal core dispersion to prepare a precursor-metal core mixed solution; b2) mixing the buffer solution with the precursor-metal core mixed solution to prepare a reaction solution and reacting the reaction solution at a temperature of 15 to 40′C, advantageously at room temperature to prepare Raman-active particles; and b3) separating and recovering the produced Raman-active particles and adding the recovered Raman-active particles to a buffer solution (a separate buffer solution) to store the solution at a temperature of 1 to 10° C., specifically at a temperature of 1 to 5° C.

By step b), the Raman-active particles including the metal core, the self-assembled monolayer of the Raman reporter surrounding the metal core, and the metal shell surrounding the self-assembled monolayer may be produced, and the Raman-active particles may have an average size of 100 nm or less, substantially 40 to 100 nm, and more substantially 60 to 100 nm.

In a specific example, the method of producing Raman-active particles may further include c) fixing a receptor which is bonded (specifically bonded) to a substance to be detected to the metal shell, after step b). Step c) may be performed by mixing the receptor with the prepared Raman-active particle dispersion solution, and fixing may be performed depending on a protocol known for each receptor, of course.

In addition, before step a), a step of washing the metal core using an organic solvent and the like, so that the spherical metal core has a bare metal surface, may be performed, but the washing is enough to be performed if necessary.

The present invention includes the Raman-active particles produced from the production method described above.

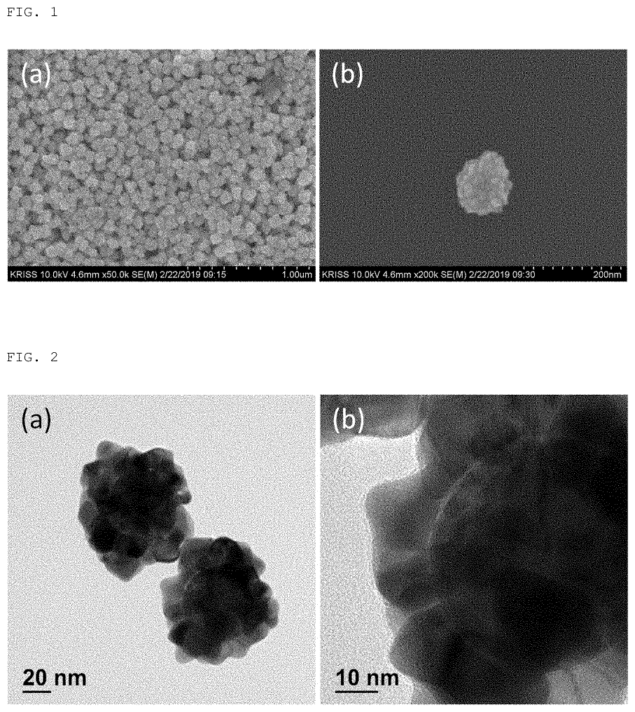

FIG. 1 is scanning electron micrographs of the Raman-active particles produced according to an exemplary embodiment of the present invention at a low magnification (a) and at a high magnification (b).

Specifically, the Raman-active particles were produced by mixing spherical Au nanoparticles (diameter=50 nm) as a metal core with 1 mL of a 1 mM bis(p-sulfonatophenyl)phenylphosphine dihydrate dipotassium salt (BSPP) solution and sonicating the solution for 10 minutes to prepare an Au core dispersion solution having a molar concentration of 0.1 nM. 1 mL of an Au core dispersion solution and 50 μL of a 1,4-benzenedithiol (BDT) solution were mixed, sonicated for 10 minutes, and then centrifuged at 6000 rpm for 10 minutes to recover an Au core on which the self-assembled monolayer of BDT which is the Raman reporter. The recovered Au core on which a self-assembled monolayer was formed was dispersed in 1 mL of deionized water (molar concentration of 0.1 nM), 500 μL of 5 mM HAuCl₄ and 2.5 mL of a 50 mM HEPES buffer solution at pH 7.2 were added to the dispersion solution, the solution was stirred at 1000 rpm for 30 minutes, and an excessive amount of deionized water was added thereto to complete the reaction. Thereafter, centrifugation was performed at 4000 rpm, 3000 rpm, and 2000 rpm for 10 minutes to prepare the Raman-active particles, and the particles were recovered, added to 1 ml of a 50 mM HEPES buffer solution, and stored.

It was confirmed from FIG. 1 that Raman-active particles having a shell formed thereon, having surface unevenness formed by Au fine particles, were produced. The produced Raman-active particles had an average diameter of 95 nm, and the Au fine particles forming the shell had an average size of 22 nm. As shown in FIG. 1, it was found that Au fine particles protruded and surface unevenness was formed on the shell, and it was confirmed that unevenness by protrusion of the fine particles was evenly formed, in all directions based on a particle center.

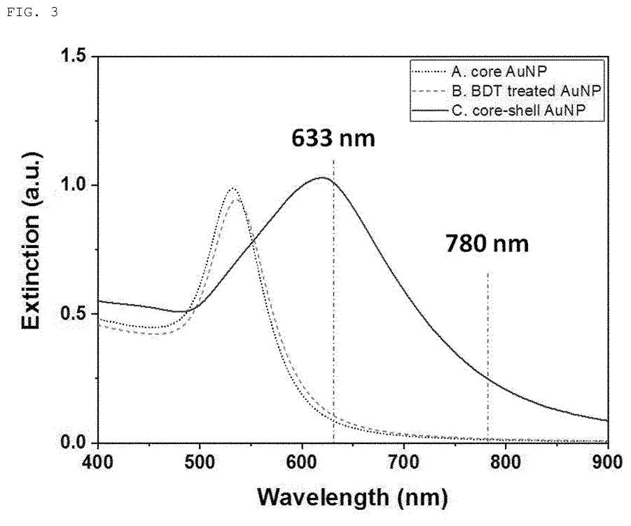

FIG. 2 is transmission electron micrographs of the Raman-active particle.

It was confirmed that the self-assembled monolayer of the Raman reporter was positioned between an Au core and an Au shell of a polycrystal composed of Au fine particles, and a nanogap having a thickness of 0.8 bn was formed in the whole area of the particle.

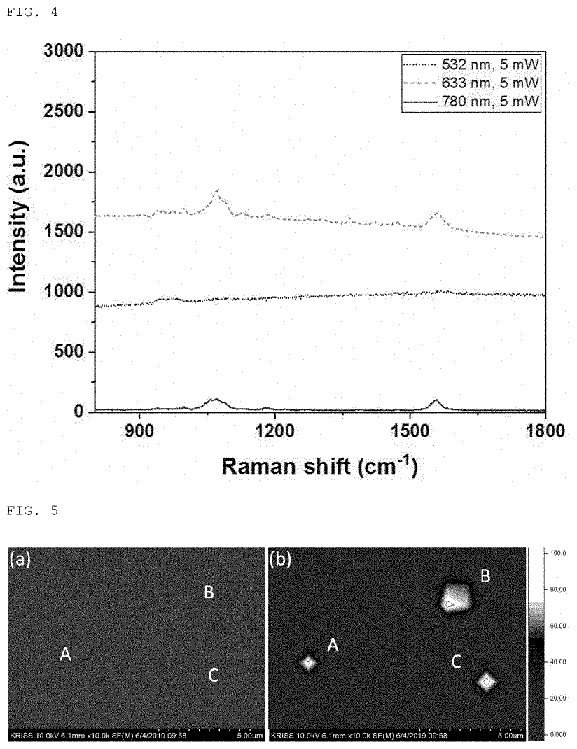

FIG. 3 is a drawing illustrating a UV-Vis absorption spectrum of each of an Au core itself (cpre AuNP in FIG. 3), an Au core on which a self-assembled monolayer is formed (BDT-treated AuNP in FIG. 3), and the produced Raman-active particles. As seen from FIG. 3, it was found that the Au core and the Au core on which the self-assembled monolayer was formed represented substantially almost similar absorption spectra, but the produced Raman-active particle had an absorption peak which was shifted to about 620 nm. In addition, unlike the Au core or the Au core on which the self-assembled monolayer was formed, the Raman-active particle had a very broad absorption peak of a half width of 200 nm or more. In addition, it was found that though the Raman-active particle had a very high absorbance around 620 nm, the particle also had a significant absorbance even at a near-infrared ray at 750 nm or more, specifically at 780 nm or more.

FIG. 4 is a drawing illustrating a Raman spectrum of measuring the produced Raman-active particles themselves, and the spectrum was obtained by irradiating a laser at 532 nm (5 mW), a laser at 633 nm (5 mW), or a laser at 780 nm (5 mW). As seen from FIG. 4, it was found that when light at 633 nm was irradiated, the highest intensity of Raman signal was obtained, but a strong Raman signal was still obtained even when near-infrared light at 780 nm was irradiated. In addition, as seen from FIG. 4, when light in a visible region was irradiated, very large basal fluorescence occurred. However, it was found that when near-infrared light at 780 nm was irradiated, any significant basal fluorescence to affect a detection signal did not occur. Thus, it was found that when Raman spectroscopic analysis was performed by irradiating the Raman-active particles according to a specific exemplary embodiment of the present invention with a near-infrared ray, the detected signal intensity may be immediately used as a Raman signal intensity without separate signal treatment, and reliable Raman analysis may be performed. As is known, a signal processing process for removing basal fluorescence may cause Raman signal distortion, which is problematic in quantitative analysis.

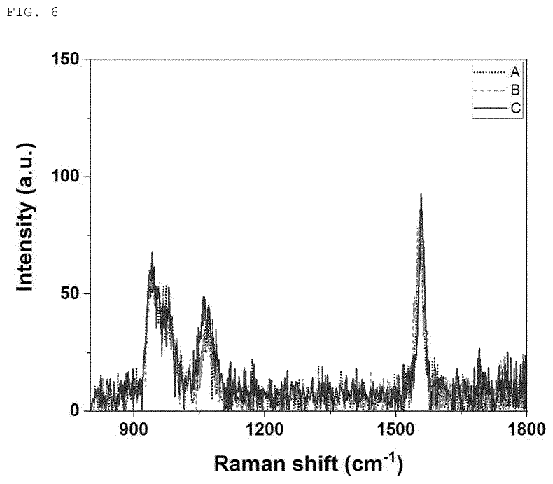

FIG. 5 is a scanning electron micrograph (a) of the Raman-active particles (A, B, and C) observed after positioning the particles on a silicon substrate, and a drawing illustrating Raman mapping (laser at 780 nm, 5 mW) (b) in an area observed by a scanning electron microscope. As seen from FIG. 5, it was found that the produced Raman-active particles had very uniform Raman activity.

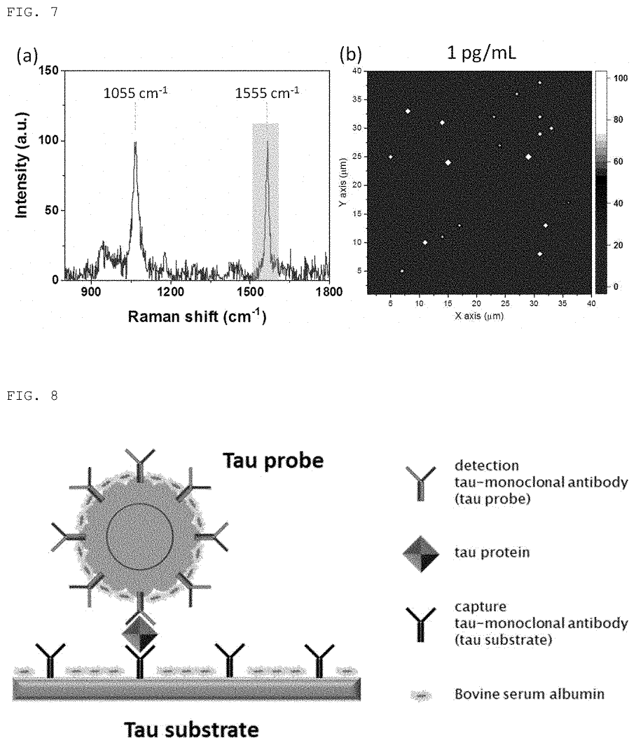

FIG. 6 is a drawing illustrating a Raman spectrum of each of the Raman-mapped Raman-active particles (A, B, and C) in FIG. 5 by overlapping. As seen from FIG. 6, it was confirmed that the substantially the same Raman spectra between the Raman-active particles were obtained.

Similarly, a Raman spectrum of each Raman-active particle was obtained for 60 Raman-active particles produced and then an average value and a standard deviation of the peak intensity for one Raman peak were measured, and as a result, the average value was 87.6 (a.u.) and the standard deviation was 7.5 (a.u.), and thus, it was confirmed therefrom that extremely uniform Raman activity between particles was shown.

FIG. 7 is drawings illustrating a Raman spectrum (a) and a Raman map (using a Raman signal of 1555 cm⁻¹) (b) obtained from tau protein as a substance to be detected, a monoclonal tau antibody having different antibody epitopes from each other as a first receptor and a second receptor, and a phosphate-buffered saline (PBS) buffer solution containing tau protein at a concentration of 1 pg/mL as a sample.

Specifically, Raman-active particles on which the first receptor was formed and a substrate on which a second receptor was formed were produced as follows: Raman-active particles on which the first receptor was formed: 1 μM 11-mercaptoundecanoic acid (MUA) was added to 1 mL of a dispersion solution of Raman-active particles (particle concentration of 1 nM) produced as shown in the observation photograph of FIG. 1, the solution was allowed to stand overnight, and then the solution was centrifuged (3000 rpm, 10 minutes) to recover particles. Thereafter, 20 μM 1-ethyl-3-(3-dimethylaminopropyl) carbodiimide (EDC) and 5 μM N-hydroxysuccinimide (NHS) were added to the recovered Raman-active particles, a reaction was performed for 15 minutes, and then centrifugation (3000 rpm, 10 minutes) was performed to recover Raman-active particles which were surface-modified with a carboxyl group. Thereafter, the Raman-active particles which were surface-modified with a carboxyl group were added at a concentration of 10 μg/mL to 1 mL of a 50 mM HEPES buffer solution (pH 7.4) containing the monoclonal tau antibody (first receptor) and a reaction was performed for 2 hours, bovine serum albumin (BSA) was added at 1 wt % to the same solution and a reaction was performed for 1 hour, and then centrifugation (3000 rpm, 10 minutes) was performed to produce Raman-active particles on which the first receptor and a blocking molecule were formed. The thus-produced Raman-active particles (hereinafter, referred to as a tau probe) were dispersed and stored in 1 mL of a 50 mM HEPES buffer solution.

Substrate on which a second receptor was formed: A substrate having a gold (Au) surface was immersed overnight in a 1 mM 11-mercaptoundecanoic acid (MUA) solution, and then washed with ethanol/deionized water. Thereafter, the recovered substrate was immersed in a solution in which 200 mM 1-ethyl-3-(3-dimethylaminopropyl) carbodiimide (EDC) and 50 mM N-hydroxysuccinimide (NHS) were mixed with deionized water to perform a reaction for 30 minutes, and then the substrate was washed with deionized water again, thereby producing a gold substrate which was surface-modified with a carboxyl group. 20 mL of a 50 mM HEPES buffer solution (pH 7.4) containing the monoclonal tau antibody (second receptor) having a different epitope from the first receptor at a concentration of 50 μg/mL was applied on the produced gold substrate which was surface-modified with a carboxyl group, and then the substrate was washed with a 1 wt % of bovine serum albumin (BSA) solution, thereby producing a substrate on which the second receptor was formed (hereinafter, referred to as tau substrate).

Sample detection was performed as follows: 0.1 mL of a sample was dropped on the produced tau substrate (substrate size=1 cm×1 cm, active surface area=1 μm×1 μm) to perform a reaction for 60 minutes, and then an unreacted sample was washed and removed using deionized water. Thereafter, 200 μL of a 50 mM HEPES buffer solution containing the tau probe at a concentration of 0.5 nM was dropped on the tau substrate reacted with the sample to perform a reaction for 1 hour, and then an unreacted tau probe was washed and removed using deionized water.

Raman analysis was performed as follows: laser excitation light at 780 nm, laser power of 5 mW, a N.A. 0.75 lens, an acquisition time of 1 second, a 1 μm mapping pitch, a mapping area size of 40 μm×40 μm, and a mapping image of a Raman signal at 1555 cm⁻¹.

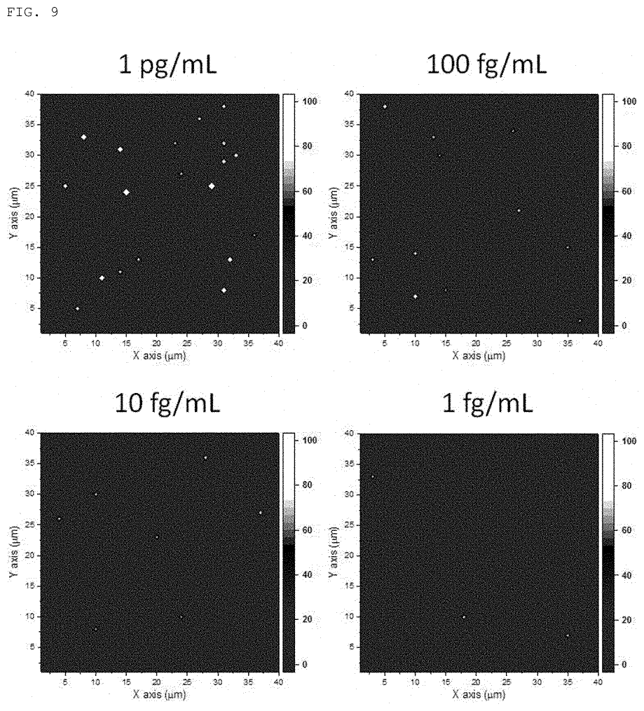

FIG. 8 is a schematic diagram illustrating a structure in which tau protein is bonded to the second receptor of the tau substrate and the tau probe is bonded to the tau protein bonded to a substrate. Like an example illustrated in FIG. 8, the substance to be detected was fixed to the active surface by the second receptor and the Raman-active particles were fixed to the substance to be detected fixed to the active surface by the first receptor. Each point (square points in FIG. 7) at which a Raman signal was detected in the Raman map in FIG. 7 corresponds to an area on which a bonding structure of active surface-substance to be detected-Raman-active particles is formed.

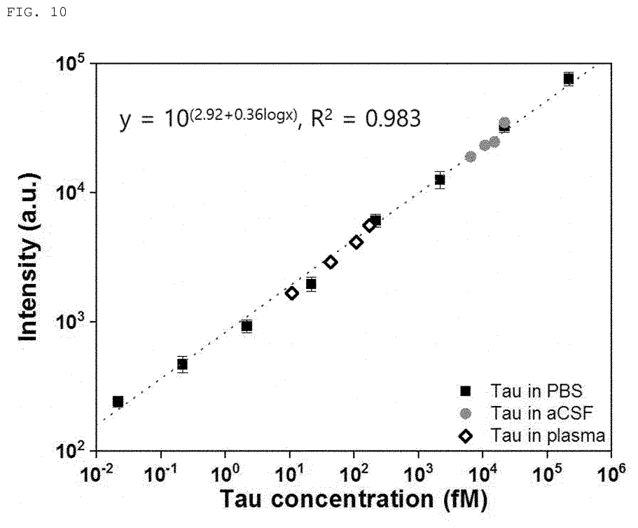

FIG. 9 is a drawing of Raman mapping results of performing the same Raman analysis using the same tau substrate and tau probe as in FIG. 7, except sampling a phosphate-buffered saline (PBS) buffer solution containing tau protein at a concentration of 1 μg/mL, 100 fg/mL, 10 fg/mL, or 1 fg/mL. As seen from FIG. 9, as the concentration of the substance to be analyzed in the sample is increased, the number of substances to be analyzed which are bonded to each of the active surface and the Raman-active particle and fixed between the active surface and the Raman-active particle is increased, and the number of points at which the Raman signal is detected in the Raman map as in FIG. 9 is increased.

FIG. 10 is a drawing illustrating a reference graph of log value-summed intensity of tau protein molar concentration (Tau in PBS in FIG. 10=a measured value of a standard sample, straight line=reference graph) obtained by analyzing a sample having a different tau protein concentration as in FIGS. 7 and 9 and results of detecting a cerebrospinal fluid (Tau in aCSF in FIG. 10) or plasma (Tau in plasma in FIG. 10) collected from a human body as a sample. Here, the summed intensity is intensity obtained by summing up maximum intensities at each point at which the Raman signal is detected in the Raman map measured using each sample, and when tau protein of the standard sample and the sample collected from the human body is detected, a Raman mapping area, Raman analysis condition (concentration, amount, and the like of the applied Raman-active particle dispersion solution), and the like were all the same.

As seen from FIG. 10, it was found from a semi-log graph of a log value—summed intensity of a molar concentration of tau protein that the log value (MC) and the summed intensity (Iₛᵤₘ) of the molar concentration of the tau protein has a linear relation of MC=aIₛᵤₘ+b (a and b are real numbers), it was found from the fact that a determination coefficient (R²) is 0.983 that extremely excellent linearity at a very wide concentration range of 10⁻² to 10⁶ fM was maintained, and it was found from a limit of detection (LOD) in a level of about 20 aM that the sensitivity was such that even one tau protein may be detected.

In addition, in FIG. 10, as a result of comparing results of quantitatively detecting a tau protein concentration in a cerebrospinal fluid sample (Tau in aCSF in FIG. 10) or a plasma sample (Tau in plasma in FIG. 10) with the tau protein concentration which was actually contained in each sample, using the reference graph, it was confirmed that the tau protein was quantitatively detected at an accuracy up to about 97%.

Hereinabove, although the present invention has been described by specific matters, limited exemplary embodiments, and drawings, they have been provided only for assisting the entire understanding of the present invention, and the present invention is not limited to the exemplary embodiments, and various modifications and changes may be made by those skilled in the art to which the present invention pertains from the description.

Therefore, the spirit of the present invention should not be limited to the above-described exemplary embodiments, and the following claims as well as all modified equally or equivalently to the claims are intended to fall within the scope and spirit of the invention.

1. A detection method comprising:
bringing Raman-active particles into contact with a sample which may contain a substance to be detected, the Raman-active particle including a spherical plasmonic metal core, a plasmonic metal shell having surface unevenness, a self-assembled monolayer which is bonded to each of the core and the shell and positioned between the core and the shell, and includes a Raman reporter, and a first receptor which is positioned on a surface of the plasmonic metal shell and may be specifically bonded to the substance to be detected; and
irradiating excitation light thereon to detect the substance to be detected in the sample.

2. The detection method of claim 1, wherein the excitation light is a near-infrared ray.

3. The detection method of claim 1, wherein
a) bringing a substrate in which a second receptor which is specifically bonded to the substance to be detected is positioned on a surface into contact with the sample;
b) bringing the substrate in contact with the sample into contact with the Raman-active particles;
c) irradiating the substrate in contact with the Raman-active particles with the excitation light to obtain a Raman spectrum; and
d) detecting the presence of the substance to be detected in the sample and a concentration of the substance to be detected, based on the Raman spectrum, are included.

4. The detection method of claim 2, wherein when the excitation light is irradiated, performing Raman mapping in a predetermined area to obtain a Raman map of one Raman signal, is included, and the presence of the substance to be detected in the sample and the concentration of the substance to be detected are detected, based on a total intensity obtained by summing up maximum intensities to the one Raman signal on the Raman map.

5. The detection method of claim 4, wherein the concentration of the substance to be detected is calculated by the following Equation 1:
MC=aIₛᵤₘ+b (Equation 1)
wherein MC is a log value of a molar concentration of the substance to be detected, Iₛᵤₘ is the total intensity, and a and b are a constant, respectively.

6. The detection method of claim 5, wherein a limit of detection (LOD) of Equation 1 is 20 aM or less.

7. The detection method of claim 3, further comprising: removing an unreacted sample after a) and removing unreacted Raman-active particles after b).

8. The detection method of claim 1, wherein the substance to be detected is one or two or more selected from lesion biomarkers having lesion specificity, pathogens, proteins, nucleic acids, sugars, drugs, and biochemical materials.

9. The detection method of claim 4, wherein basal fluorescence is not removed from the Raman map.

10. The detection method of claim 1, wherein the plasmonic metal shell includes plasmonic metal fine particles having an average size of 0.1D to 0.6D, based on a diameter (D) of the metal core, and has the surface unevenness due to the plasmonic metal fine particles.

11. The detection method of claim 10, wherein in the plasmon metal shell, an inner shape of the shell in contact with the self-assembled monolayer is spherical.

12. The detection method of claim 1, wherein the self-assembled monolayer has a thickness of 0.5 to 2.0 nm.

13. A detection method of a lesion marker for disease diagnosis, the method comprising:
bringing a substrate in which a second receptor which is specifically bonded to the lesion marker is positioned on a surface into contact with a living body-derived sample which may contain the lesion marker;
bringing the substrate in contact with the living body-derived sample into contact with Raman-active particles and then washing the substrate, the Raman-active particle including a spherical plasmonic metal core, a plasmonic metal shell having surface unevenness, a self-assembled monolayer which is bonded to each of the core and the shell and positioned between the core and the shell, and includes a Raman reporter, and a first receptor which is positioned on a surface of the plasmonic metal shell and may be specifically bonded to a substance to be detected;
irradiating the substrate in contact with the Raman-active particles with near-infrared excitation light and performing Raman mapping in a predetermined area to obtain a Raman map for the one Raman signal; and
detecting the presence of the lesion marker in the living body-derived sample and a concentration of the lesion marker, based on a total intensity obtained by summing up maximum intensities to the one Raman signal on the Raman map.

14. A detection device comprising:
a surface-enhanced Raman scattering-active reagent including Raman-active particles which include a spherical plasmonic metal core, a plasmonic metal shell having surface unevenness, a self-assembled monolayer which is bonded to each of the core and the shell and positioned between the core and the shell, and includes a Raman reporter, and a first receptor which is positioned on a surface of the plasmonic metal shell and may be specifically bonded to a substance to be detected; and
a substrate in which a second functional group which is specifically bonded to a substance to be detected is positioned on a surface.
